(19) United States
(12) Patent Application Publication
(10) Pub. No.: US 2002/0198160 A1
(43) Pub. Date: Dec. 26, 2002

(54) Compositions and methods for enhancing the bioavailability of pharmaceutical agents

(76) Inventors: Elizabeth A. Everitt, Libertyville, IL (US); Edward K. Han, Gurnee, IL (US); Sajeev P. Cherian, Lake Bluff, IL (US); Dale J. Kempf, Libertyville, IL (US); Hing L. Sham, Vernon Hills, IL (US); Shi-Chung Ng, Libertyville, IL (US)

Correspondence Address: ABBOTT LABORATORIES, DEPT. 377 - AP6D-2, 100 ABBOTT PARK ROAD, ABBOTT PARK, IL, 60064-6050, US

(21) Appl. No.: 10/134,931

(22) Filed: Apr. 29, 2002

Related U.S. Application Data
(60) Provisional application No. 60/367,353, filed on May 1, 2001.

(51) Int. Cl. A61K 31/7056

(52) U.S. Cl. 514/43

(57) ABSTRACT

This invention relates to enhancing the bioavailability of pharmaceutically active agents. In particular, this invention relates to the use of lopinavir, its pharmaceutically acceptable equivalents, and derivatives thereof as P-glycoprotein inhibitors.

This application claims priority to provisional application Serial No. 60/367,353 which was filed on May 1, 2001.

### FIELD OF THE INVENTION

This invention relates to enhancing the bioavailability of pharmaceutically active agents. In particular, this invention relates to the use of lopinavir as a P-glycoprotein inhibitor.

### BACKGROUND OF THE INVENTION

Enhancement of the bioavailability of a pharmaceutically active agent can provide a more efficient and effective treatment for patients because, for a given dose, more of the pharmaceutically active agent will be available at the targeted tissue sites. Bioavailability is the degree to which the pharmaceutically active agent becomes available to the target tissue after the agent's introduction into the body. In some cases, poor bioavailability of pharmaceutically active agents is caused by the activity of a multidrug transporter, such as membrane-bound P-glycoprotein. P-glycoprotein functions as an energy-dependent efflux pump to decrease the intracellular accumulation of some pharmaceutically active agents. It is believed that P-glycoprotein limits the ability of certain pharmaceutically active agents to transverse cells and be absorbed into the body's systemic circulation. This lack of absorption can reduce the overall bioavailability of the active agents.

More specifically, it is believed that P-glycoprotein facilitates the reverse transport of substances out of a cell that have diffused into or have been transported into the cell. For example, it is believed that P-glycoprotein in the intestinal epithelial cells may function as a protective efflux pump that limits toxic substances that have been ingested and have diffused or have been transported into the cells from being absorbed into the circulatory system and becoming bioavailable. One potentially negative aspect of this function of P-glycoprotein is that it can prevent substances that are beneficial, such as certain pharmaceutically active agents, from diffusing into or being transported into cells and the bloodstream.

P-glycoprotein is expressed in a variety of types of epithelial and endothelial cells including tissues such as those in the adrenal cortex, the intestine, the brush border of the proximal renal tubule epithelium, the secretory endothelium (such as the biliary lining of the bile duct), the pancreatic ductules, and the vascular endothelial cells lining the brain, placenta and testis.

P-glycoprotein is also found in membranes of cancerous tumor cells. Many anticancer pharmaceutically active agents have poor bioavailability due to the expression of P-glycoprotein by tumor cells. Tumor cells from patients undergoing chemotherapy often exhibit elevated levels of P-glycoprotein, which enhance multidrug resistance. Multidrug resistance in cancer is defined as the condition of a tumor cell in which the cell is resistant to various unrelated anticancer drugs such as adriamycin, daunomycin, vinblastine, vincristine, paclitaxel, actinomycin D, and etoposide, after being exposed to only one of these types of pharmaceutically active agents. It is thought that the exposure of tumor cells to a cytotoxic agent, such as a pharmaceutically active anticancer agent, causes the increased expression of P-glycoprotein. P-glycoprotein mediates a reverse transport system in the tumor cell membrane that pumps many anticancer agents, along with other broad classes of cytotoxic agents, out of the tumor cell causing multiple drug resistance for the cell.

It is feasible that the administration of an effective amount of a pharmaceutically active agent along with a P-glycoprotein inhibitor would enhance the bioavailability of various pharmaceutically active agents. Reduction of the activity of the P-glycoprotein transport system tends to cause fewer molecules of the pharmaceutically active agent to be transported out of the cells and can increase the net transport of the pharmaceutically active agent into the bloodstream, and thus, ultimately increase the number of molecules available to effect the desired change in the target tissues.

### SUMMARY OF THE INVENTION

It has been discovered that lopinavir is a P-glycoprotein inhibitor. Thus, by administering lopinavir along with one or more pharmaceutically active agents, the bioavailability of the pharmaceutically active agent(s) can be enhanced.

The invention is directed toward methods for inhibiting P-glycoprotein which comprise administering lopinavir to a mammal (e.g., a human) in need of such treatment. Typically, lopinavir is administered as a part of a pharmaceutical composition. The pharmaceutical composition may also include one or more pharmaceutically active and/or other P-glycoprotein-inhibiting agents, such as ritonavir or a therapeutically acceptable salt thereof.

Methods for enhancing the bioavailability of pharmaceutically active agents are also described. These methods comprise co-administering to a mammal, preferably a human, in need of such treatment a pharmaceutically active agent and lopinavir. Lopinavir and/or the pharmaceutically active agent may be administered as a part of one or more pharmaceutical compositions. Any such pharmaceutical composition may also contain ritonavir or a therapeutically acceptable salt thereof. Ritonavir may also be otherwise co-administered with lopinavir and the pharmaceutically active agent.

Methods for increasing the central nervous system penetration of a pharmaceutically active agent are also described herein. These methods comprise co-administering to a mammal, preferably a human, in need of such treatment a pharmaceutically active agent and lopinavir. The pharmaceutically active agent(s) and/or lopinavir can be administered as part of one or more pharmaceutical compositions. Any such pharmaceutical composition may contain ritonavir or a therapeutically acceptable salt thereof. Ritonavir may also be otherwise co-administered with lopinavir and the pharmaceutically active agent.

Methods for increasing the absorption of a pharmaceutically active agent from the gastrointestinal tract are also described. These methods comprise co-administering to a mammal, preferably a human, in need of such treatment the pharmaceutically active agent and lopinavir. One or both of the pharmaceutically active agent and lopinavir may be administered as a part of one or more pharmaceutical compositions. Optionally, ritonavir or a therapeutically acceptable salt thereof can be included in any such pharmaceutical composition. Ritonavir may also be otherwise co-administered with lopinavir and the pharmaceutically active agent.

Also disclosed are methods for treating multidrug resistance. These methods comprise co-administering to a mammal, such as a human, in need of such treatment a pharmaceutically active agent to treat the multidrug resistance and lopinavir. The pharmaceutically active agent and/or lopinavir may be administered as a part of one or more pharmaceutical compositions. Any such pharmaceutical compositions may also contain ritonavir, or a therapeutically acceptable salt thereof. Ritonavir may also be otherwise co-administered with lopinavir and the pharmaceutically active agent.

The invention is also directed toward methods of treating cancer. In particular, such methods comprise co-administering to a mammal, preferably a human, in need of such treatment an anticancer agent and lopinavir. One or both of the pharmaceutically active agent and lopinavir may be administered as part of one or more pharmaceutical compositions. Any such pharmaceutical composition may further comprise ritonavir, or a therapeutically acceptable salt thereof. Ritonavir may also be otherwise co-administered with lopinavir and the pharmaceutically active agent.

Besides cancer, the invention is also directed toward treating a viral infection, particularly HIV, in mammals (e.g., humans). Specifically, the method comprises administering to a mammal in need of such treatment an antiviral agent and lopinavir. One or both of the antiviral agent and lopinavir may be administered as part of one or more pharmaceutical compositions. Any such compositions may also contain ritonavir, or a therapeutically acceptable salt thereof. Ritonavir may also be otherwise co-administered with lopinavir and the pharmaceutically active agent.

The invention also describes pharmaceutical compositions useful for treating cancer. In particular, the invention is directed toward a pharmaceutical composition comprising lopinavir and a pharmaceutically active anticancer agent. Preferably, the anticancer agent is a taxane, spindle poison, epidophylloptoxin, or an antibiotic. More preferably, the anticancer agent is paclitaxel. The compositions may also include ritonavir, or a therapeutically acceptable salt thereof. Ritonavir may also be otherwise co-administered with lopinavir and the pharmaceutically active agent.

### BRIEF DESCRIPTION OF THE DRAWING

FIG. 1 is a graphical representation of the rate of efflux of paclitaxel, ritonavir, and lopinavir from HCT15 cells.

### DETAILED DESCRIPTION OF THE INVENTION

As used herein, the singular forms “a”, “an”, and “the” include plural reference unless the context clearly dictates otherwise. The terms “pharmaceutically active agent(s)” and “drug(s)” are used interchangeably herein. As used herein, the following terms have the meanings identified below.

The term “antiviral agent” refers to an agent useful in the treatment of a viral infection (e.g. Human Immunodeficiency Virus, or HIV). Examples of antiviral agents include, but are not limited to, acyclic nucleosides (e.g., acyclovir, valaciclovir, famciclovir, ganciclovir, and penciclovir), protease inhibitors (e.g., ritonavir, indinavir, nelfinavir, saquinavir, and amprenavir), reverse transcriptase inhibitors (e.g., dideoxycytidine (ddC; zalcitabine), dideoxyinosine (ddI; didanosine), BCH-189, ddA, d4C, d4T (stavudine), 3TC (lamivudine), 3′-azido-3′-deoxythymidine (AZT), (2R,5S)-5-fluoro-1-[2-(hydroxymethyl)-1,3-oxathiolan-5-yl]cytosine (FTC), and abacavir), interferons such as α-interferon, and non-nucleoside reverse transcriptase inhibitors (e.g., nevirapine, efavirenz, and delavirdine).

The term “bioavailability” refers to the degree and rate at which a pharmaceutically active agent, or other substance, becomes available to a target tissue within a mammal.

The terms “chemotherapeutic agent” and “anticancer agent” refer to therapies useful in the treatment of cancer. Examples of chemotherapeutic agents include, but are not limited to, taxanes such as paclitaxel or docetaxel; alkylating agents such as cyclophosphamide, isosfamide, melphalan, hexamethylmelamine, thiotepa or dacarbazine; antimetabolites such as pyrimidine analogues, for instance 5-fluorouracil and cytarabine or its analogues such as 2-fluorodeoxycytidine or folic acid analogues such as methotrexate, idatrexate or trimetrexate; spindle poisons including vinca alkaloids such as vinblastine or vincristine or their synthetic analogues such as navelbine, or estramustine or taxoids; epidophylloptoxins such as etoposide or teniposide; antibiotics such as daunorubicin, doxorubicin, bleomycin or mitomycin; enzymes such as L-asparaginase; topoisomerase inhibitors such as camptothecin derivatives (i.e., rubitecan, CPT-11, and topotecan) or pyridobenzoindole derivatives; farnesyl transferase inhibitors; matrix metalloproteinase inhibitors; TSP analogs; and various agents such as procarbazine, mitoxantrone, E-7010, leuprolide, platinum coordination complexes such as cisplatin or carboplatin; and biological response modifiers or growth factor inhibitors such as interferons or interleukins. Illustrative examples of cancers include cutaneous tumors, such as malignant melanomas and mycosis fungoids; hematologic tumors such as leukemias, for example, acute lymphoblastic, acute myelocytic or chronic myelocytic leukemia; lymphomas, such as Hodgkin's disease or malignant lymphoma; gynecologic tumors, such as ovarian and uterine tumors; urologic tumors, such as those of the prostate, bladder, or testis; soft tissue sarcomas, osseous or non-osseous sarcomas, breast tumors; tumors of the pituitary, thyroid and adrenal cortex; gastrointestinal tumors, such as those of the esophagus, stomach, intestine, and colon; pancreatic and hepatic tumors; laryngeal papillomestasas and lung tumors.

The terms “co-administer(s)”, “co-administering”, and “co-administration” all refer to with respect to compounds or compositions, administering substantially simultaneously one or more compounds or compositions, for example, administering one or more P-glycoprotein-inhibiting compounds with one or more pharmaceutically active agents, such as, but not limited to, those agents included in antiviral therapy or anticancer therapy. “Substantially simultaneously” means that the compound (e.g., lopinavir ) is typically administered during or within a reasonably short time either before or after the administration of other compounds, such as a pharmaceutically active agent that treats the disease in question. Additionally, “co-administration”, “co-administer(s)”, and “co-administering” include administering more than one dose of the pharmaceutically active agent within 24 hours after a dose of P-glycoprotein inhibitor. In other words, P-glycoprotein inhibitor(s) need not be administered again before or with every administration of a pharmaceutically active agent, but may be administered intermittently during the course of treatment. “Co-administration”, “co-administer(s)”, and “co-administering” also include administering a pharmaceutically active agent and a P-glycoprotein inhibitor (e.g., lopinavir) as a part of one or more pharmaceutical compositions, and such one or more pharmaceutical compositions may contain a co-formulation of a P-glycoprotein inhibitor and a pharmaceutically active agent or individual formulations of a pharmaceutically active agent and a P-glycoprotein inhibitor.

The term “P-glycoprotein inhibitor” refers to an organic compound that inhibits or reduces the activity of the P-glycoprotein-mediated transport system. Various P-glycoprotein inhibitors are well known and appreciated in the art. These include water soluble vitamin E; polyethylene glycol; poloxamers including Pluronic F-68; polyethylene oxide; polyoxyethylene castor oil derivatives, cyclosporin A (also known as cyclosporine), verapamil, tamoxifen, quinidine, ritonavir, indinavir, nelfinavir, saquinavir, amprenavir, and phenothiazines.

The term “pharmaceutically active” when referencing one or more agents means any one or more medicaments except lopinavir.

The term “lopinavir” refers to a pharmaceutically active agent represented by the chemical name [1S-[1R*,(R*),3R*,4R*]]-N-[4-[[(2,6-dimethylphenoxy)acetyl]amino]-3-hydroxy-5-phenyl-1-(phenylmethyl)pentyl]tetrahydro-alpha-(1-methylethyl)-2-oxo-(2H)-pyrimidine acetamide, which is shown structurally below, its pharmaceutically acceptable equivalents, pharmaceutical derivatives, and pharmaceutical analogs as described in U.S. Pat. No. 5,914,332, which issued on Jun. 22, 1999 and is hereby incorporated by reference.

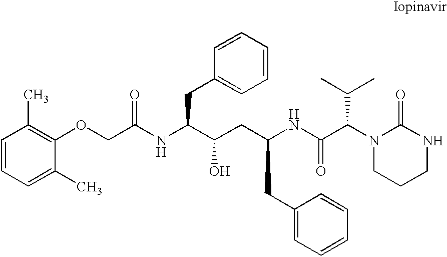

The term “multidrug resistance” refers to a specific type of drug resistance characterized by cross-resistance to more than one functionally and/or structurally unrelated drugs. Multidrug resistance can be either intrinsic or acquired.

The term “ritonavir” refers to a pharmaceutically active agent represented by the chemical name [5S-(5R*,8R*,10R*,11R*)]-10-hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic acid, 5-thiazolylmethyl ester, which is shown below, its pharmaceutically acceptable equivalents, therapeutically acceptable salts, pharmaceutical derivatives, and pharmaceutical analogs as described in U.S. Pat. No. 5,541,206, which issued on Jul. 30, 1996 and is hereby incorporated by reference.

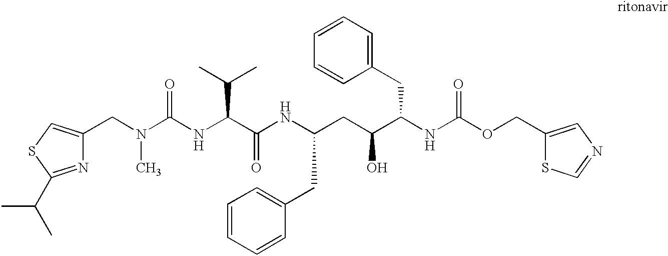

The term “substrate” refers to a compound that binds to P-glycoprotein and is subsequently effluxed out of the cell. In determining whether a compound acts as a substrate, paclitaxel, a known P-glycoprotein substrate, is used as a comparison. By one criterion, if the amount of compound effluxed out of the cell is comparable to or higher than the amount of paclitaxel effluxed out of the cell, under similar conditions, then the compound is considered herein to be a substrate.

The term “therapeutically effective amount” or “therapeutically effective dose” refers to a sufficient amount of a compound or composition to treat disorders at a reasonable benefit/risk ratio. It is understood that the total daily usage of the compounds and compositions of the present invention will be decided by the attending physician within the scope of sound medical judgment. The specific therapeutically effective dose for any particular patient tends to depend upon a variety of factors including, but not limited to: the type and severity of the disorder being treated; the level and type of activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. It is understood by one of skill in the art that one may start doses of the compound at levels lower than required to achieve the desired therapeutic effect and gradually increase the dosage until the desired effect is achieved.

The term “therapeutically acceptable salt” refers to salts or zwitterionic forms of the compounds of the present invention that are water or oil-soluble or dispersible, that are suitable for treatment of diseases without undue toxicity, irritation, and allergic response, that are commensurate with a reasonable benefit/risk ratio, and that are effective for their intended use. The salts can be prepared during the final isolation and purification of the compounds or separately, for example, by reacting an amino group with a suitable acid. Representative acid addition salts include, but are not limited to, acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isothionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention may be quaternized with: (1) methyl, ethyl, propyl, and butyl chlorides, bromides, and iodides; (2) dimethyl, diethyl, dibutyl, and diamyl sulfates; (3) decyl, lauryl, myristyl, and steryl chlorides, bromides, and iodides; and (4) benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

The invention is directed toward enhancement of pharmaceutically active agents' bioavailability by inhibiting P-glycoprotein. In particular, the invention describes the use of lopinavir in an effective amount to inhibit P-glycoprotein. The invention is also directed toward methods of inhibiting P-glycoprotein and of increasing the bioavailability of various pharmaceutically active agents.

Lopinavir

Lopinavir is a known HIV protease inhibitor. Applicants have discovered that lopinavir is also an inhibitor of P-glycoprotein. It is believed that inhibitors of P-glycoprotein may or may not be substrates of P-glycoprotein, and it appears that lopinavir is not a substrate of P-glycoprotein. In other words, it is believed that lopinavir does not readily bind to the P-glycoprotein in such a manner that it is easily transported out of a cell by the P-glycoprotein transport mechanism. On the other hand, ritonavir, which is also believed to be a P-glycoprotein inhibitor, is believed to be a substrate for the protein, because it is easily transported out of the cell by the P-glycoprotein transport mechanism. For more information about P-glycoprotein substrates, see, for example, Drewe, J., et. al. Biochemical Pharmacology 1999, 57, 1147-1152; and Lee, C. G. L., et. al. Biochemistry 1998, 37, 3594-3601. It is believed that both types of compounds (i.e., those that are P-glycoprotein substrates and those that are not) can be inhibitors of P-glycoprotein, but that the mechanisms of inhibition could differ between these two types of compounds.

Inhibitors of P-glycoprotein have been shown to increase the penetration into the central nervous system of HIV protease inhibitors, such as indinavir, saquinavir, nelfinavir, ritonavir, and amprenavir. Inhibitors of P-glycoprotein may also be used to enhance absorption of HIV protease inhibitors from the gastrointestinal tract and to enhance penetration into other P-glycoprotein expressing tissues such as lymphocytes, testis, kidney, liver, and placenta. Enhanced absorption of HIV protease inhibitors from the gastrointestinal tract, for example, may result in reduced oral dosages, toxicity, and side effects for patients in need of such treatment.

It is known that the administration of a P-glycoprotein inhibitor with an anticancer agent improves the bioavailability of the anticancer agent. For example, published PCT application WO97/15269 (published May 1, 1997) teaches that the combination of paclitaxel with cyclosporin A, a known P-glycoprotein inhibitor, achieves local tissue concentrations when dosed orally that are comparable to those obtained when paclitaxel alone was administered intravenously. Thus, lopinavir being a P-glycoprotein inhibitor can also be used in the treatment of cancer by co-administering it with a known anticancer drug such as paclitaxel.

The amount of lopinavir to be co-administered with a pharmaceutically active agent to a patient tends to depend upon the patient, the disease state being treated, the severity of the affliction, the manner and schedule of administration, and the pharmaceutically active agent to be co-administered with the lopinavir. Lopinavir should not be administered in amounts that would reduce the effectiveness of the pharmaceutically active agent. In addition, lopinavir should be administered to patients in an amount sufficient to inhibit P-glycoprotein. When lopinavir is simultaneously administered with the pharmaceutically active agent, the amount of lopinavir that is useful may be reduced when compared with an administration scheme in which the lopinavir is administered before the pharmaceutically active agent. Total daily dose administered to a mammalian host, preferably a human, in single or divided doses may be in amounts, for example, from 0.001 to 300 mg/kg body weight daily. In a preferred range, lopinavir is administered in amounts of 0.1 to 1600 mg/day. Dosage unit compositions may contain sufficient amounts of submultiples thereof to make up the daily dose. Generally, depending on the intended mode of administration, a pharmaceutical composition may contain from about 0.005% to about 95%, preferably from about 0.5% to about 50%, by weight of lopinavir; and the remainder of the composition may include one or more suitable pharmaceutical excipients, carriers, diluents, and/or pharmaceutically active agents.

Ritonavir is known to enhance the bioavailability of lopinavir because ritonavir is believed to inhibit cytochrome P450 monooxygenase, which is an enzyme that may metabolize lopinavir and/or pharmaceutically active agents in the liver. The most preferred co-administration of lopinavir is with ritonavir (see U.S. Pat. No. 6,037,157, issued Mar. 14, 2000, which is hereby incorporated by reference). Preferred co-formulations of lopinavir and ritonavir are disclosed in published PCT patent application WO98/22106, published May 28, 1998; and U.S. patent application Ser. No. 09/576,097, filed May 22, 2000, which are hereby incorporated by reference.

Pharmaceutically Active Agents

In accordance with the invention, pharmaceutically active agents are co-administered with lopinavir. Any type of pharmaceutically active agent, the effectiveness or bioavailability of which is reduced by the P-glycoprotein transport mechanism, is useful as a pharmaceutically active agent in the invention. Typical pharmaceutically active agents include chemotherapeutic agents and antiviral agents.

Examples of chemotherapeutic agents believed to be useful in the invention include, but are not limited to, taxanes such as paclitaxel or docetaxel; alkylating agents such as cyclophosphamide, isosfamide, melphalan, hexamethylmelamine, thiotepa or dacarbazine; antimetabolites such as pyrimidine analogues, for instance 5-fluorouracil and cytarabine or its analogues such as 2-fluorodeoxycytidine or folic acid analogues such as methotrexate, idatrexate or trimetrexate; spindle poisons including vinca alkaloids such as vinblastine or vincristine or their synthetic analogues such as navelbine, or estramustine or taxoids; epidophylloptoxins such as etoposide or teniposide; antibiotics such as daunorubicin, doxorubicin, bleomycin or mitomycin; enzymes such as L-asparaginase; topoisomerase inhibitors such as camptothecin derivatives (i.e., rubitecan, CPT-11, and topotecan) or pyridobenzoindole derivatives; farnesyl transferase inhibitors; matrix metalloproteinase inhibitors; TSP analogs; and various agents such as procarbazine, mitoxantrone, E-7010, leuprolide, platinum coordination complexes such as cisplatin or carboplatin; and biological response modifiers or growth factor inhibitors such as interferons or interleukins.

Examples of antiviral agents believed to be useful in the invention include, but are not limited to, acyclic nucleosides (e.g., acyclovir, valaciclovir, famciclovir, ganciclovir, and penciclovir), protease inhibitors (e.g., ritonavir, indinavir, nelfinavir, saquinavir, and amprenavir), reverse transcriptase inhibitors (e.g., dideoxycytidine (ddC; zalcitabine), dideoxyinosine (ddI; didanosine), BCH-189, ddA, d4C, d4T (stavudine), 3TC (lamivudine), 3′-azido-3′-deoxythymidine (AZT), (2R,5S)-5-fluoro-1-[2-(hydroxymethyl)-1,3-oxathiolan-5-yl]cytosine (FTC), and abacavir), interferons such as α-interferon, and non-nucleoside reverse transcriptase inhibitors (e.g., nevirapine, efavirenz, and delavirdine).

The amount of pharmaceutically active agent to be administered to patients in need of treatment varies with the amount of P-glycoprotein inhibitor that is administered, the disease being treated, and the overall health condition of the patient. When the pharmaceutically active agent is co-administered with the P-glycoprotein inhibitor, the dosage of the pharmaceutically active agent may be reduced depending upon how much the bioavailability of the pharmaceutically active agent is enhanced by the P-glycoprotein inhibitor. The total daily dose of a pharmaceutically active agent administered to a mammalian host, preferably a human, may be in single or divided doses. Dosage unit compositions may contain such amounts of submultiples thereof to make up the total daily dose. The process for determining the amount of pharmaceutically active agent to be administered is well known in the art, and the amount of pharmaceutically active agent to be administered should be determined by a physician.

A preferred pharmaceutically active agent that is useful in conjunction with lopinavir is paclitaxel. Applicants have discovered that adding lopinavir (5 or 10 μM concentrations) to paclitaxel leads to a 9- and >37-fold decrease, respectively, in the IC₅₀ of paclitaxel in HCT15 cells (which express P-glycoprotein). Co-administration of cyclosporin A, a known P-glycoprotein inhibitor, and paclitaxel caused a >37 fold decrease in the IC₅₀ of paclitaxel.

In addition, treatment of paclitaxel resistant H460/T800 cells (which over express P-glycoprotein) with combinations of lopinavir (5 or 10 μM) and paclitaxel resulted in a 11- and 21-fold reduction of the IC₅₀ of paclitaxel, respectively. These results indicate that lopinavir, acting as a P-glycoprotein inhibitor, is useful in the treatment of multidrug resistance in diseases such as cancer.

The above data indicates that lopinavir inhibits P-glycoprotein's ability to efflux paclitaxel, thereby increasing paclitaxel's potency, even in cells that show resistance to treatment with paclitaxel alone (i.e., in H460/T800 cells). Thus, co-administration of lopinavir and an anticancer agent, preferably paclitaxel, can treat patients afflicted with benign and malignant tumors or neoplasms, including melanomas, lymphomas, leukemias, and sarcomas. Tumors that typically are or become multidrug resistant can be treated with the compounds and methods of this invention. Such tumors include, but are not limited to, colon tumors, lung tumors, stomach tumors, and liver tumors.

When administering lopinavir and/or any pharmaceutically active agent(s) in accordance with the invention, any pharmaceutically acceptable mode of administration can be used. The lopinavir and/or pharmaceutically active agent(s) can be administered either alone or in combination with other pharmaceutically acceptable excipients. These pharmaceutically acceptable excipients include, but are not limited to, solid, semi-solid, and liquid dosage forms, such as, for example, tablets, capsules, powders, liquids, suspensions, suppositories, or the like. Lopinavir and/or the pharmaceutically active agent(s) in accordance with the invention can also be administered in sustained or controlled release dosage forms, including depot injections, osmotic pumps, pills, transdermal (including electrotransport) patches, and the like, for the prolonged administration of the compound at a predetermined rate, preferably in unit dosage forms suitable for single administration of precise dosages. Co-formulated compositions can typically include a pharmaceutically active agent, a conventional pharmaceutical carrier, diluent or excipient and the P-glycoprotein inhibitor(s) or therapeutically acceptable equivalents thereof. The excipient(s) must be “acceptable” in the sense of being compatible with the other ingredients of the composition and not deleterious to the recipient thereof. In addition, these compositions may include other medicinal agents, pharmaceutical agents, carriers, adjuvants, etc., such as the anticancer and the antiviral therapeutics listed above.

For oral administration, a convenient daily dosage regimen which can be adjusted according to the degree of affliction can be used. For such oral administration, a pharmaceutically acceptable, non-toxic composition is formed by incorporating into the composition with lopinavir and/or the pharmaceutically active agent any of the normally employed excipients, such as, for example, mannitol, lactose, starch, magnesium stearate, sodium saccharine, talcum, cellulose, sodium crosscarmellose, glucose, gelatin, sucrose, magnesium carbonate, and the like. The term “composition(s)” as used herein includes, but is not limited to, solutions, suspensions, tablets, dispersible tablets, pills, capsules, powders, sustained release formulations, and the like.

Preferably oral compositions will take the form of a liquid or solid pill, tablet, or capsule. Thus, a pharmaceutical composition in accordance with the invention may contain along with the active ingredient(s) a diluent such as lactose, sucrose, dicalcium phosphate, or the like; a lubricant such as magnesium stearate or the like; and a binder such as starch, gum acacia, gelatin, polyvinylpyrrolidone, cellulose and derivatives thereof, and the like.

Liquid pharmaceutically active compositions can, for example, be prepared by dissolving, dispersing, etc., a pharmaceutically active agent, lopinavir, and optional pharmaceutical adjuvants in a carrier, such as, for example, water, saline, mannitol, aqueous dextrose, glycerol, glycol, ethanol, and the like, to thereby form a solution or suspension. If desired, the pharmaceutical composition may also contain minor amounts of non-toxic auxiliary substances such as wetting agents, emulsifying agents, solubilizing agents, pH buffering agents and the like. Some examples of these types of substances include but are not limited to, acetate, sodium citrate, cyclodextrin derivatives, sorbitan monolaurate, triethanolamine sodium acetate, triethanolamine oleate, etc. Actual methods of preparing such dosage forms are known, or are apparent to those skilled in the art.

Parenteral administration is generally characterized by injection (e.g., subcutaneously, intramuscularly, intravenously) or infusion through a central line. P-glycoprotein inhibitors (e.g., lopinavir) and pharmaceutically active agents can be administered parenterally as injectables and can be prepared in conventional forms, either as liquid solutions or suspensions, solid forms suitable for solution or suspension in liquid prior to injection, or as emulsions. Suitable excipients for such injectable forms are, for example, water, saline, dextrose, glycerol, ethanol, mannitol, or the like. In addition, if desired, pharmaceutical compositions to be administered may also contain minor amounts of non-toxic auxiliary substances such as wetting or emulsifying agents, pH buffering agents, solubility enhancers, and the like. Examples include sodium acetate, sorbitan monolaurate, triethanolamine oleate, and cyclodextrins. Parenteral administration can also include the implantation of a slow-release or sustained-release system so that a constant level of dosage is maintained.

The following examples serve to further illustrate, without limiting, the novel use of lopinavir as a P-glycoprotein inhibitor.

### EXAMPLES

Lopinavir's ability to inhibit P-glycoprotein is shown in the group of examples below entitled “Transepithelial Bi-Directional Transport Studies Across Caco-2 Cells”, (i.e., the results of which are summarized in Tables 1A and 1B). This ability is demonstrated by measuring the apparent permeability of the pharmaceutically active agent vinblastine across cell membranes exhibiting P-glycoprotein when the agent is administered in combination with a P-glycoprotein inhibitor. As shown in these tables, vinblastine's ability to permeate the cell monolayer improves significantly when it is co-administered with cyclosporin A or ritonavir, known P-glycoprotein inhibitors, or lopinavir.

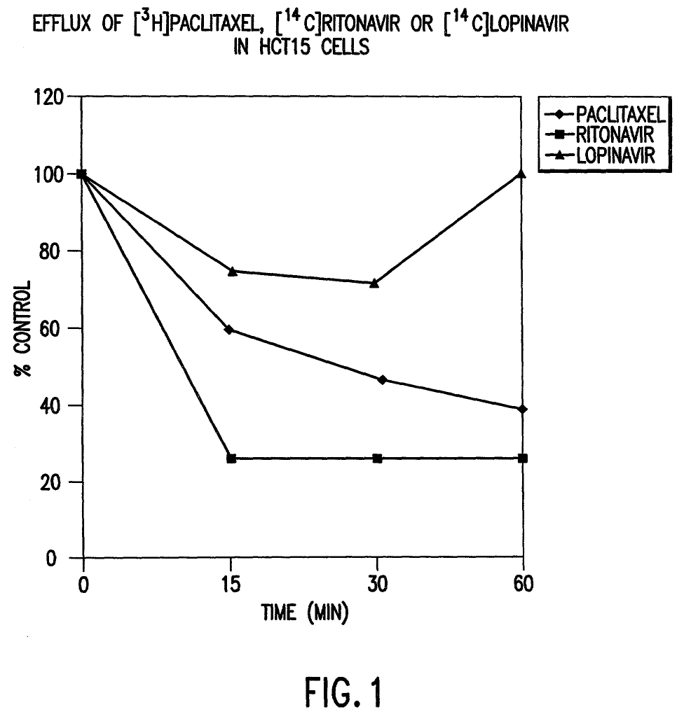

The group of examples entitled “Lopinavir and Ritonavir Transport Studies” (i.e., the results of which are summarized in Tables 2A and 2B) show that although ritonavir and lopinavir are both P-glycoprotein inhibitors, ritonavir also behaves as a substrate for the protein, while lopinavir does not. This data is corroborated by FIG. 1, entitled “Efflux of [³H]Paclitaxel, [¹⁴C]Ritonavir or [¹⁴C]Lopinavir in HCT15 Cells”, which also shows ritonavir's ability to serve as a P-glycoprotein substrate and lopinavir's inability to serve as a substrate.

Transepithelial Bi-Directional Transport Studies Across Caco-2 Cells

In order to determine the apparent permeability of lopinavir and ritonavir with and without cyclosporin A and of vinblastine with and without cyclosporin A across Caco-2 cell membranes, Caco-2 intestinal cancer cells expressing P-glycoprotein were obtained from American Tissue Culture Collection (ATCC) of Rockville, Md. The cells were grown and maintained in DMEM (Dulbecco's modified eagle's medium, purchased from Gibco/BRL, Grand Island, N.Y.) supplemented with 10% fetal bovine serum and 2 mM L-glutamine.

Cell cultures were maintained, antibiotic free, in a 37° C. incubator with 90% relative humidity in an atmosphere of 5% CO₂. The cells were maintained in 10 cm² stock plates and seeded onto polycarbonate Transwell™ HTS filter inserts at 4×10⁵ cells/mL. The cells were cultured on the filters for about 27-30 days. Caco-2 cells having passages between 40 and 110 were used for the studies.

The integrity of the cell monolayers was determined by measuring the paracellular transepithelial transport of the integrity marker, Lucifer yellow, which is commercially available from Sigma Chemical of St. Louis, Mo. The rate of Lucifer yellow transport in Caco-2 cell monolayers used in these experiments was generally <0.25%. The rate of Lucifer yellow transport was determined by using the procedures disclosed in the article entitled “The Use of Cultured Epithelial and Endothelial Cells for Drug Transport and Metabolism Studies”, Pharmacol. Res. 1990:7(5) 435-451, which is hereby incorporated by reference.

Vinblastine Transport Studies

For the vinblastine apparent permeability studies, after being cultured on the filters, the cell monolayers were rinsed with HBSS (Hank's buffered saline solution purchased from Gibco/BRL, Grand Island, N.Y.) at a pH of 7.4 and pre-incubated for 30 minutes at a temperature of 37° C. with HBSS and the P-glycoprotein inhibitors cyclosporin A, ritonavir, and lopinavir, as noted in Tables 1A and 1B below. At the beginning of each experiment, HBSS containing vinblastine (5 μM) radiolabelled with tritium was applied to either the apical side (AP), which has a pH of 6.8, or the basolateral (BL) side, which has a pH of 7.4, of the cell monolayers either alone or in combination with ritonavir (10 μM), lopinavir (10 μM), or cyclosporin A (10 μM). It is understood that P-glycoprotein is expressed primarily on the AP side of the cell monolayers. The transport of vinblastine, which is the pharmaceutically active agent in these studies, was allowed to proceed for 120 minutes at 37° C. Stock concentrations (C₀) of each of the P-glycoprotein inhibitors were treated similarly as dosed cells and were retained and sampled for starting dose. Aliquots for both the AP and the BL sides of the cell monolayers were taken at 2 hours, and such aliquots were analyzed by liquid scintillation counting (LSC) using a Packard Liquid Scintillation Counting machine. Calculation of the apparent permeability was done by using the following Artusson equation:

$$P ( a p p ) = \frac{ⅆ Q \times 1}{ⅆ T {C}_{0} \times A} ;$$

where dQ/dT is the flux rate (μg/sec), C₀ is concentration of the starting material at t=0 (μg/mL), and A=the area of the monolayer (cm²). For the triplicate wells the P(app) is the mean of the three individual P(app) values for each well. These values were incorporated into the data for calculation of the overall P(app) as detailed in Tables 1A and 1B below.

TABLE 1A
Transepithelial Bi-Directional Transport
Studies Across Caco-2 Cells (Run 1)*
P(app) (1 × 10(−6) cm/sec ± SD)

| | | | Ratio |
|---|---|---|---|
| Compound | AP-BL | BL-AP | (BL-AP/AP-BL) |
| Vinblastine | 0.91 ± 0.07 | 17.73 ± 0.39 | 19.48 |
| Vinblastine + CyA | 2.16 ± 0.10 | 7.08 ± 0.80 | 3.27 |
| Vinblastine + RVR | 1.95 ± 0.10 | 8.73 ± 0.53 | 4.47 |
| Vinblastine + LVR | 2.54 ± 0.47 | 9.56 ± 1.52 | 3.76 |
| *The rates of AP-BL and BL-AP transport, and inhibition of transport of vinblastine (5 μM) in the presence or absence of cyclosporin A (CyA, 10 μM), ritonavir (RVR, 10 μM), or lopinavir (LVR, 10 μM) was determined after 120 minutes incubation at 37° C. Vinblastine was added to the donor or receiver compartment and its appearance in the opposite compartment was followed over time. The transport and inhibited transport values are expressed as apparent permeability (P(app)), | | | |
| #wherin the triplicate well values are expressed as a mean value ± standard deviation. | | | |

TABLE 1B
Transepithelial Bi-Directional Transport
Studies Across Caco-2 Cells (Run 2)*
P(app) (1 × 10(−6) cm/sec ± SD)

| | | | Ratio |
|---|---|---|---|
| Compound | AP-BL | BL-AP | (BL-AP/AP-BL) |
| Vinblastine | 0.49 ± 0.00 | 14.71 ± 3.33 | 30.02 |
| Vinblastine + CyA | 3.40 ± 0.33 | 5.87 ± 0.41 | 1.73 |
| Vinblastine + RVR | 2.55 ± 0.46 | 9.49 ± 0.31 | 3.72 |
| Vinblastine + LVR | 2.63 ± 0.14 | 6.62 ± 0.29 | 2.52 |
| *The rates of AP-BL and BL-AP transport, and inhibition of transport of vinblastine (5 μM) in the presence or absence of cyclosporin A (CyA, 10 μM), ritonavir (RVR, 10 μM), or lopinavir (LVR, 10 μM) was determined after 120 minutes incubation at 37° C. Vinblastine was added to the donor or receiver compartment and its appearance in the opposite compartment was followed over time. The transport and inhibited transport values are expressed as apparent permeability (P(app)), | | | |
| #wherin the triplicate well values are expressed as a mean value ± standard deviation. | | | |

The results shown in Tables 1A and 1B indicate that both ritonavir and lopinavir act as active P-glycoprotein inhibitors in the presence of vinblastine. When vinblastine alone is administered to the AP side of the cell monolayers, the apparent permeability is significantly lower than when administered to the BL side. These results demonstrate the ability of P-glycoprotein, which is primarily expressed on the AP side, to inhibit the passage of the vinblastine. When vinblastine is administered to the AP side of the cell monolayer along with a P-glycoprotein inhibitor, such as cyclosporin A, ritonavir, or lopinavir, the apparent permeability increases relative to when the vinblastine is administered alone. Due to the inhibition of the P-glycoprotein efflux pump transport mechanism, there is reduced efflux of the vinblastine back out to the apical medium, resulting in an increase in permeability from the AP side to the BL side. When co-administered with a P-glycoprotein inhibitor to the BL side, the permeability is decreased relative to when the vinblastine is applied alone. It is believed that this decrease is related to the absence of the efflux pump, which results in the inhibition of the active transport of the vinblastine from the BL to the AP medium, causing a relative decrease in the apparent permeability.

The results from these tables show that lopinavir serves as a P-glycoprotein inhibitor and facilitates the ability of a pharmaceutically active agent to permeate cells.

Lopinavir and Ritonavir Transport Studies

For the lopinavir and ritonavir apparent permeability studies, after being cultured on the filters, the cell monolayers were rinsed with HBSS at a pH of 7.4 and pre-incubated for 30 minutes at a temperature of 37° C. with HBSS and lopinavir, lopinavir with cyclosporin A, ritonavir, or ritonavir with cyclosporin A, as noted in Tables 2A and 2B below. For each experiment, the cell monolayers were incubated for 120 minutes at a temperature of 37° C. with HBSS plus the one or more other ingredients, as noted in Tables 2A and 2B below (i.e., lopinavir (5 μM), lopinavir (5 μM) with cyclosporin A (10 μM) ritonavir (5 μM), and ritonavir (5 μM) with cyclosporin A (10 μM)).

The lopinavir, lopinavir with cyclosporin A, ritonavir, or ritonavir with cyclosporin A (collectively known as “P-glycoprotein inhibitors”) was applied to the AP side of the cell monolayers or the BL side of the cell monolayers, as noted in Tables 2A and 2B below. Stock concentrations (C₀) of each of the P-glycoprotein inhibitors were treated similarly as dosed cells and were retained and sampled for starting dose. Aliquots for both the AP and the BL sides of the cell monolayers were taken at 2 hours, and such aliquots were analyzed by LSC Calculation of the apparent permeability by using the Artusson equation shown above. For the triplicate wells the P(app) is the mean of the three individual P(app) values for each well. These values were incorporated into the data for calculation of the overall P(app) as detailed in Tables 2A and 2B.

TABLE 2A
Lopinavir and Ritonavir Transport Studies (Run 1)*
P(app) (1 × 10(−6) cm/sec ± SD)

| | | | Ratio |
|---|---|---|---|
| Compound | AP-BL | BL-AP | (BL-AP/AP-BL) |
| Lopinavir | 10.65 ± 0.58 | 9.55 ± 2.10 | 0.90 |
| Lopinavir + CyA | 24.10 ± 1.03 | 9.69 ± 1.50 | 0.40 |
| Ritonavir | 10.97 ± 1.32 | 15.15 ± 1.37 | 1.38 |
| Ritonavir + CyA | 13.27 ± 0.43 | 11.22 ± 0.54 | 0.85 |
| *The rates of AP-BL and BL-AP transport, and inhibition of transport of lopinavir (5 μM) and ritonavir (5 μM) in the presence or absence of cyclosporin A (CyA, 10 μM), was determined after 120 minutes incubation at 37° C. Lopinavir and ritonavir were added to the donor or receiver compartment and its appearance in the opposite compartment was followed over time. The transport and inhibited transport values are expressed as apparent permeability (P(app)), wherein the | | | |
| #triplicate well values are expressed as a mean value ± standard deviation. | | | |

TABLE 2B
Lopinavir and Ritonavir Transport Studies (Run 1)*
P(app) (1 × 10(−6) cm/sec ± SD)

| | | | Ratio |
|---|---|---|---|
| Compound | AP-BL | BL-AP | (BL-AP/AP-BL) |
| Lopinavir | 18.72 ± 1.04 | 17.42 ± 4.37 | 0.93 |
| Lopinavir + CyA | 20.55 ± 1.34 | 12.40 ± 0.65 | 0.60 |
| Ritonavir | 8.95 ± 0.87 | 19.15 ± 0.70 | 2.14 |
| Ritonavir + CyA | 18.95 ± 0.85 | 15.82 ± 2.06 | 0.83 |
| *The rates of AP-BL and BL-AP transport, and inhibition of transport of lopinavir (5 μM) and ritonavir (5 μM) in the presence or absence of cyclosporin A (CyA, 10 μM), was determined after 120 minutes incubation at 37° C. Lopinavir and ritonavir were added to the donor or receiver compartment and its appearance in the opposite compartment was followed over time. The transport and inhibited transport values are expressed as apparent permeability (P(app)), wherein the | | | |
| #triplicate well values are expressed as a mean value ± standard deviation. | | | |

The results shown in Tables 2A and 2B indicate that lopinavir is not a substrate for P-glycoprotein. The apparent permeability (P(app)) of lopinavir is about the same regardless of whether the lopinavir is applied to the AP or the BL side of the cell, demonstrating that the P-glycoprotein on the AP side is not hindering lopinavir's ability to permeate the monolayer. When lopinavir is applied along with cyclosporin A, a known P-glycoprotein inhibitor, the ratio of BL/AP permeability does not significantly change, generally, indicating that lopinavir is not a substrate of P-glycoprotein, and may not benefit from the co-administration of an additional P-glycoprotein inhibitor.

Conversely, the data in Tables 2A and 2B show that ritonavir is a substrate for P-glycoprotein. Ritonavir's apparent permeability is significantly lower when applied to the AP side than it is when applied to the BL side. As the AP side is the side which primarily expresses P-glycoprotein, this data suggests that the ritonavir is behaving as a substrate for the P-glycoprotein, inhibiting ritonavir's ability to pass through the monolayer. In contrast to lopinavir, ritonavir does benefit from co-administration with the known P-glycoprotein inhibitor cyclosporin A. As shown in Tables 2A and 2B, ritonavir's ratio of BL/AP permeability is significantly lowered in the presence of cyclosporin A, indicating that its ability to serve as a substrate of P-glycoprotein is hindered when the protein is inhibited.

These results indicate that while both lopinavir and ritonavir are P-glycoprotein inhibitors, ritonavir also serves as a substrate for the protein, while lopinavir does not.

Lopinavir, Ritonavir, and Paclitaxel Efflux Studies

For the measurement of drug efflux, 1×10⁶ HCT15 cells (which express P-glycoprotein) which were purchased from American Tissue Culture Collection (ATCC) of Rockville, Md.) were plated in 35-mm culture dishes and maintained in RPMI medium (Roswell Park Memorial Institute medium, available from Life Technologies, Rockville, Md. with 10% fetal bovine serum for 16 hours. The medium was aspirated and fresh medium containing either [³H]paclitaxel (5 μM, 0.5 μCi/ml), [¹⁴C]ritonavir (1 μM, 0.03 μCi/ml), or [¹⁴C]lopinavir (1 μM, 0.15 μCi/ml) was added for 1 hour. The medium was aspirated and the cells were washed with PBS (phosphate buffered saline, available from Life Technologies, Rockvile, Md.). Serum-free RPMI medium was added and the cells were collected at the times indicated in FIG. 1. At harvest, the cells were washed once with PBS and the pellets were dissolved in 550 μl of 1N NaOH. The total protein was determined by Bio-Rad assay (available from Bio-Rad of Hercules, Ca.) and the total radioactivity was determined by LSC. Following normalization of the protein amount, cpm/μg protein for each time point was compared to the peak drug time 0.

FIG. 1 corroborates the data shown in Tables 2A and 2B, demonstrating that lopinavir is not a substrate of P-glycoprotein. In this particular study, HCT15 cells (which express P-glycoprotein) were treated with either ritonavir or lopinavir or paclitaxel, a known P-glycoprotein substrate, which was used as a control. As shown in FIG. 1, a large amount of the ritonavir was rapidly eliminated by the HCT15 cells in 15 minutes, indicating that ritonavir serves as a substrate for P-glycoprotein, and thus is rapidly effluxed out of the cells. In contrast to ritonavir and paclitaxel, approximately 80% of the lopinavir remained in the HCT15 cells after 60 minutes. The relatively low efflux of the lopinavir by the cells indicates that in comparison to ritonavir and paclitaxel, lopinavir is not a substrate for P-glycoprotein.

It will be evident to one skilled in the art that the invention is not limited to the foregoing illustrative examples, and that it can be embodied in other specific forms without departing from the essential attributes thereof. It is therefore desired that the examples be considered in all respects as illustrative and not restrictive, reference being made to the appended claims, and all changes which come within the meaning and range of equivalency of the claims are therefore intended to be embraced therein.

What is claimed is:

1. A method for inhibiting P-glycoprotein comprising administering to a mammal in need of such treatment an amount of lopinavir effective to inhibit P-glycoprotein.

2. The method of claim 1 wherein the step of administering comprises administering a pharmaceutical composition comprising lopinavir.

3. The method of claim 1 further comprising administering ritonavir or a therapeutically acceptable salt thereof.

4. The method of claim 3 wherein the step of administering ritonavir comprises administering a pharmaceutical composition comprising ritonavir.

5. The method of claim 1, wherein the mammal is a human.

6. A method for enhancing the bioavailability of a pharmaceutically active agent comprising co-administering to a mammal in need of such treatment a combination of an amount of lopinavir effective to inhibit P-glycoprotein and a therapeutically effective amount of the pharmaceutically active agent.

7. The method of claim 6 wherein the step of co-administering comprises administering at least one pharmaceutical composition comprising lopinavir.

8. The method of claim 6, wherein the step of co-administering further comprises administering ritonavir or a therapeutically acceptable salt thereof.

9. The method of claim 8 wherein the step of administering ritonavir comprises administering at least one pharmaceutical composition comprising ritonavir or a therapeutically acceptable salt thereof.

10. The method of claim 6 wherein the mammal is a human.

11. A method for increasing the central nervous system penetration of a pharmaceutically active agent comprising co-administering to a mammal in need of such treatment a combination of an amount of lopinavir effective to inhibit P-glycoprotein and a therapeutically effective amount of the pharmaceutically active agent.

12. The method of claim 11 wherein the step of co-administering comprises administering at least one pharmaceutical composition comprising lopinavir.

13. The method of claim 11 wherein the step of co-administering further comprises administering ritonavir or a therapeutically acceptable salt thereof.

14. The method of claim 13 wherein the step of administering ritonavir comprises administering at least one pharmaceutical composition comprising ritonavir or a therapeutically acceptable salt thereof.

15. The method of claim 11 wherein the mammal is a human.

16. A method for increasing absorption of a pharmaceutically active agent from a gastrointestinal tract comprising co-administering to a mammal in need of such treatment a combination of an amount of lopinavir effective to inhibit P-glycoprotein and a therapeutically effective amount of the pharmaceutically active agent.

17. The method of claim 16 wherein the step of co-administering comprises administering at least one pharmaceutical composition comprising lopinavir.

18. The method of claim 16 further comprising administering ritonavir or a therapeutically acceptable salt thereof.

19. The method of claim 18 wherein the step of administering ritonavir comprises administering at least one pharmaceutical composition comprising ritonavir or a therapeutically acceptable salt thereof.

20. The method of claim 16 wherein the mammal is a human.

21. A method for treating multidrug resistance comprising co-administering to a mammal in need of such treatment a combination of an amount of lopinavir effective to inhibit P-glycoprotein and a therapeutically effective amount of a pharmaceutically active agent useful to treat the multidrug resistance.

22. The method of claim 21 wherein the step of co-administering comprises administering at least one pharmaceutical composition comprising lopinavir.

23. The method of claim 21 wherein the step of co-administering further comprises administering ritonavir or a therapeutically acceptable salt thereof.

24. The method of claim 23 wherein the step of administering ritonavir comprises administering at least one pharmaceutical composition comprising ritonavir or a therapeutically acceptable salt thereof.

25. The method of claim 21 wherein the mammal is a human.

26. A method for treating cancer comprising co-administering to a mammal in need of such treatment a combination of an amount of lopinavir effective to inhibit P-glycoprotein and a therapeutically effective amount of an anticancer agent.

27. The method of claim 26 wherein the step of co-administering comprises administering at least one pharmaceutical composition comprising lopinavir.

28. The method of claim 26 further comprising co-administering ritonavir or a therapeutically acceptable salt thereof.

29. The method of claim 28 wherein the step of administering ritonavir comprises administering at least one pharmaceutical composition comprising ritonavir or a therapeutically acceptable salt thereof.

30. The method of claim 26 wherein the mammal is a human.

31. A method for treating a viral infection comprising co-administering to a mammal in need of such treatment a combination of an amount of lopinavir effective to inhibit P-glycoprotein and a therapeutically effective amount of an antiviral agent.

32. The method of claim 31 wherein the step of co-administering comprises administering at least one pharmaceutical composition comprising lopinavir.

33. The method of claim 31 wherein the step of co-administering further comprises administering ritonavir or a therapeutically acceptable salt thereof.

34. The method of claim 33 wherein the step of administering ritonavir comprises administering at least one pharmaceutical composition comprising ritonavir or a therapeutically acceptable salt thereof.

35. The method of claim 31 wherein the mammal is a human.

36. The method of claim 31 wherein the viral infection is HIV.

37. A pharmaceutical composition useful for treating cancer in a mammal comprising lopinavir and a pharmaceutically active anticancer agent.

38. The pharmaceutical composition of claim 37 further comprising ritonavir or a therapeutically acceptable salt thereof.

39. The pharmaceutical composition of claim 37 wherein the anticancer agent is a taxane.

40. The pharmaceutical composition of claim 39 wherein the taxane is paclitaxel.

41. The pharmaceutical composition of claim 37 wherein the anticancer agent is a spindle poison.

42. The pharmaceutical composition of claim 37 wherein the anticancer agent is an epidophylloptoxin.

43. The pharmaceutical composition of claim 37 wherein the anticancer agent is an antibiotic.
